(19) United States
(12) Patent Application Publication
(10) Pub. No.: US 2007/0060843 A1
(43) Pub. Date: Mar. 15, 2007

(54) Method for lancing a target site with applied pressure sensing

(76) Inventors: Manuel Alvarez-Icaza, Inverness (GB); Derek McRiner, Inverarnie (GB); Steven Alexander Syme, Inverness (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/214,655

(22) Filed: Aug. 29, 2005

(51) Int. Cl. A61B 5/00; A61M 5/20

(52) U.S. Cl. 600/583; 604/137

(57) ABSTRACT

A lancing method includes contacting a contact surface of a cap body distal end with a target site, the cap body distal end being included in a cap for a lancing device. The cap body is subsequently urged towards the target site such that a portion of the contact surface moves from a first position to a second position under a predetermined force, thereby creating a target site bulge is created within an opening of the cap body. A sensor of the cap is then employed to detect that the portion of the contact surface is in the second position and the sensor signals the lancing device upon such detection. The target site bulge is thereafter lanced with the lancing device.

### BACKGROUND OF THE INVENTION

1. Field of the Invention

This invention relates, in general, to medical devices, medical kits and associated methods, and, in particular, to caps for lancing devices, lancing kits and lancing methods.

2. Description of the Related Art

A variety of medical conditions, such as diabetes, call for the monitoring of an analyte concentration (e.g., glucose concentration) in a blood, interstitial fluid or other bodily fluid sample. Typically, such monitoring requires the extraction of a bodily fluid sample from a target site (e.g., a dermal tissue target site on a user's finger). The extraction (also referred to as “expression”) of a bodily fluid sample from the target site generally involves lancing the dermal tissue target site with a lancing device and applying pressure in the vicinity of the lanced site to express the bodily fluid sample.

Conventional lancing devices generally have a rigid housing and a lancet that can be armed and launched so as to protrude from one end of the lancing device. For example, conventional lancing devices can include a lancet that is mounted within a rigid housing such that the lancet is movable relative to the rigid housing along a longitudinal axis thereof. Typically, the lancet is spring loaded and launched, upon release of the spring, to penetrate (i.e., “lance”) a target site (e.g., a dermal tissue target site). A biological fluid sample (e.g., a whole blood sample or interstitial fluid (ISF) sample) can then be expressed from the penetrated target site for collection and analysis. Conventional lancing devices are described in, for example, U.S. Pat. No. 5,730,753 to Morita, U.S. Pat. No. 6,045,567 to Taylor et al. and U.S. Pat. No. 6,071,250 to Douglas et al., each of which is incorporated fully herein by reference.

Lancing devices often include a cap with a distal end that engages the target site during use. Such a cap usually has an aperture (i.e., opening), through which the lancet protrudes during use. When a cap is engaged (i.e., contacted) with a target site, pressure is usually applied to the target site prior to launch of the lancet. This pressure urges the cap against the target site with the intent of creating a target site bulge within the opening of the cap. The lancet is then launched to penetrate the target site bulge. A biological fluid sample, typically blood, is thereafter expressed from the lanced target site bulge. The expressed biological fluid sample can then, for example, be tested for an analyte (such as glucose, lactate, ketones and HbA1c) using an associated meter.

However, conventional caps may not serve to reliably produce an adequate volume of biological fluid sample due to insufficient contact between the cap and the target site and/or inadequate application of pressure on the target site by the cap. Furthermore, in order to obtain a sufficient volume of biological fluid sample, additional pressure (such as a pumping or milking action) usually must be applied either manually or mechanically to the target site following lancing. This additional pressure can serve to facilitate expression of an adequate volume of biological fluid sample. Examples of mechanical devices designed for such use are described in co-pending U.S. application Ser. No. 10/653,023 (published as US 2004/0249253 on Dec. 9, 2004), U.S. application Ser. No. 10/652,464 (published as US 2004/0253736 on Dec. 16, 2004) and U.S. Pat. No. 5,951,493, each of which is fully incorporated herein by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a simplified perspective view of a cap for a lancing device according to an exemplary embodiment of the invention;

FIG. 2 is a combined simplified cross-sectional and partial enlarged view of the cap of FIG. 1;

FIGS. 3A through 3C are a sequence of simplified cross-sectional views of a portion of the cap of FIG. 1 being urged against a target site with a predetermined pressure;

FIG. 4 is a simplified perspective view of a cap a lancing device according to a further exemplary embodiment of the invention;

FIG. 5 is a combined simplified cross-sectional and partial enlarged view of the cap of FIG. 4;

FIG. 6 is a simplified cross-sectional view of a portion of the cap of FIG. 4 urged against a target site with a predetermined pressure; and

FIG. 7 is a flow diagram depicting stages in a process for lancing a target site according to an exemplary embodiment of the present invention

### DETAILED DESCRIPTION OF PREFERRED EMBODIMENTS

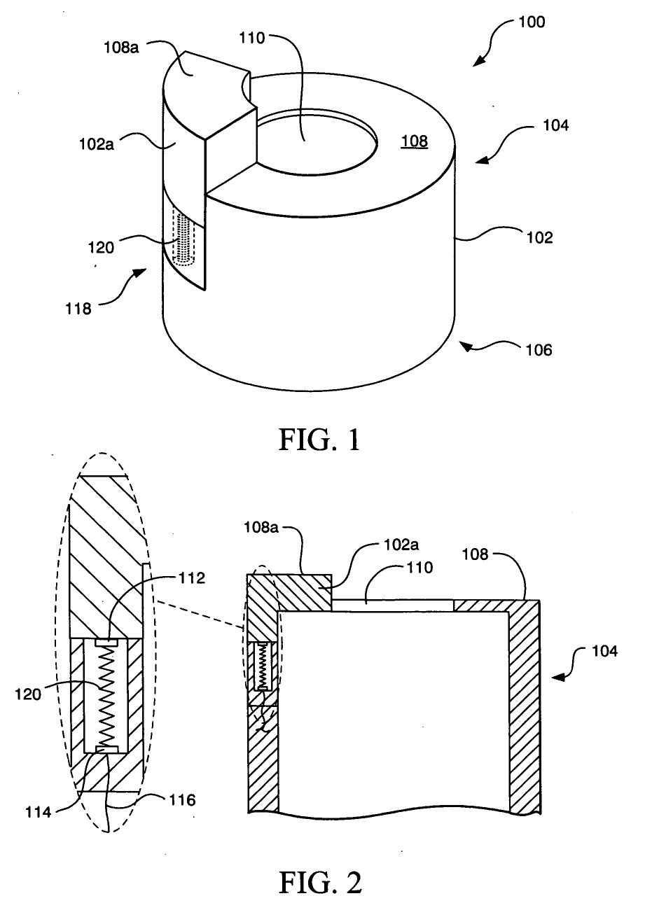

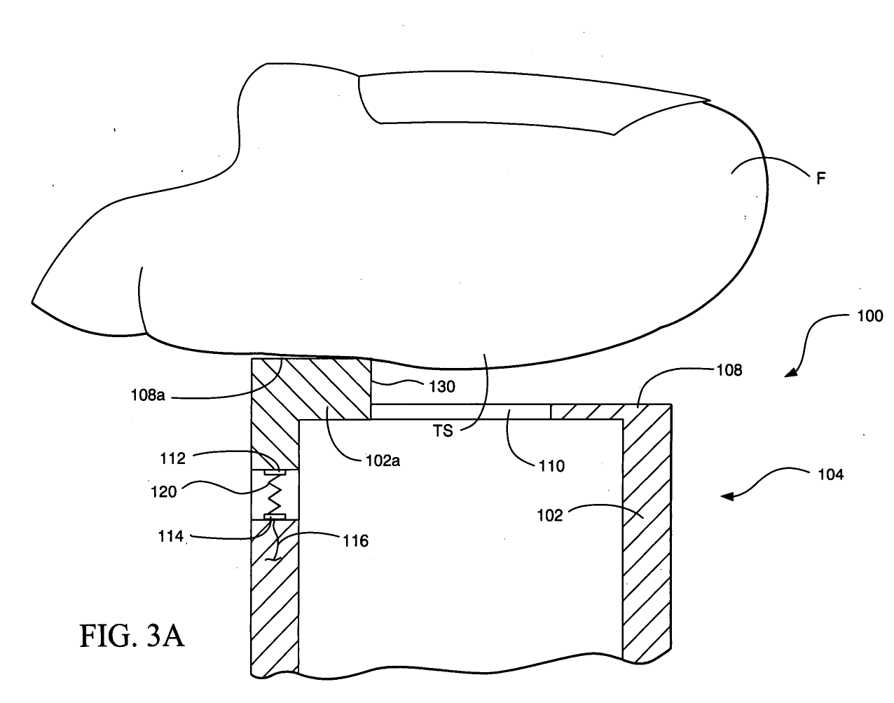

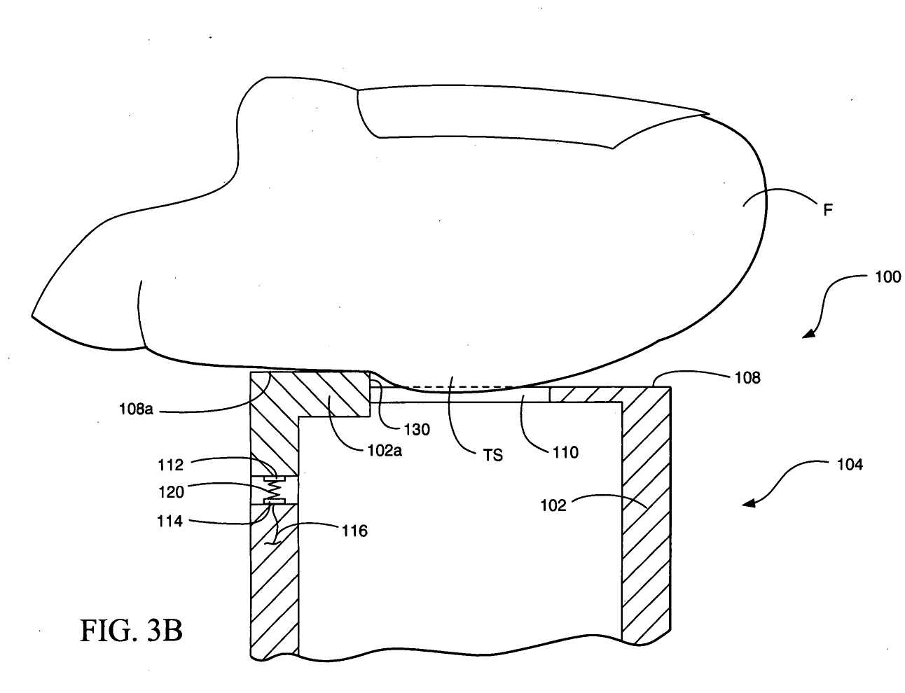

FIG. 1 is a simplified perspective view of a cap 100 for a lancing device (not shown in FIG. 1) according to an exemplary embodiment of the present invention. FIG. 2 is a combined simplified cross-sectional and partial enlarged view of the distal end of cap 100 and FIGS. 3A, 3B and 3C are a sequence of simplified cross-sectional views of cap 100 urged against a target site with a predetermined pressure (force).

Once apprised of the present disclosure, one skilled in the art will recognize that a variety of conventional lancing devices can be readily modified for use with caps according to the embodiments of the present invention, including, for example, lancing devices described in U.S. Pat. Nos. 5,730,753, 6,045,567 and 6,071,250, each of which is hereby incorporated in full by reference. Moreover, embodiments of caps according to the present invention can be employed with lancing devices that utilize various techniques for expressing a biological fluid sample from a dermal tissue target site including, but not limited to, techniques that employ lancets, hollow needles, solid needles, micro-needles, ultrasonic extraction devices, or thermal extraction devices. Furthermore, caps according to embodiments of the present invention can be employed with a combined lancing device and integrated meter for testing an analyte (e.g., a meter for testing blood glucose). In addition, such caps can be configured for urging against a dermal tissue target site of a user's finger.

Referring to FIGS. 1, 2 and 3A-3C, cap 100 includes a cap body 102 with a distal end 104 and a proximal end 106. Cap body 102 includes a moveable cap body portion 102a and distal end 104 includes a target site contact surface 108. Moveable cap body portion 102a extends beyond the remainder of cap body 102 when in a first position (see FIGS. 2 and 3A in particular). Such extension can be in the range of, for example, 5 mm to 10 mm. Proximal end 106 can be configured for attachment to a lancing device (for example, to a housing of a lancing device) by a snap fit, frictional fit or other suitable attachment technique.

Cap body 102 is depicted as primarily cylindrical in overall form. However, once apprised of the present disclosure, one skilled in the art will recognize that cap bodies employed in embodiments of the present invention can take any suitable form including, but not limited to, forms that include contoured contact surfaces and a contact surface in the form of saddle-shaped compression surface as described in U.S. patent application Ser. No. 11/045,542. Moreover, any suitable compression surface known to those of skill in the art can be employed as a contact surface in embodiments of caps for lancing devices according to the present invention, including those described in U.S. patent application Ser. No. 10/706,166, which is fully incorporated herein by reference.

Cap body 102 can be formed of any suitable material including, for example, a rigid material such as acrylonitrile butadiene styrene plastic, injection moldable plastic, polystyrene and metallic materials or a relatively resiliently deformable material, including, but not limited, to elastomeric materials, polymeric materials, polyurethane materials, latex materials, silicone materials and any combinations thereof.

Cap body 102 has an opening (i.e., aperture) 110 therethrough that extends from proximal end 106 to distal end 104. Cap 100 further includes a sensor consisting of electrical contact pads 112 and 114 and electrical signal wire 116. One skilled in the art will recognize that the entire length of electrical signal wire is not depicted in FIG. 2 or FIGS. 3A-3C.

Target site contact surface 108 of distal end 104 includes a surface portion 108a (also referred to as a “moveable” surface portion) that is moveable between a first position (depicted in FIGS. 1 and 2) and a second position (depicted in FIG. 3C) upon application of a predetermined pressure to surface portion 108a. Referring to FIGS. 2 and 3A through 3C, it should be noted that surface portion 108a is a surface of moveable cap body portion 102a of cap body 102 and that moveable cap body portion 102a travels within a guide recess 118 of cap body 102. Surface portion 108a can, for example, be a sector in the range of 30 degrees to 180 degrees of the circumference of cap 100.

Cap 100 further includes a spring 120 with a predetermined spring constant. Spring 120 is disposed between moveable cap body portion 102a and the remainder of cap body 102 within guide recess 118. Spring 120 is configured and adapted such that the force of spring 120 must be overcome to move moveable cap body portion 102a (and surface portion 108a) from the fist position of FIGS. 1 and 2 to the second position of FIG. 3C (with such movement being depicted by the sequence of FIGS. 3A through 3C). In other words, spring 120 serves to resiliently bias moveable cap body portion 102a and moveable surface portion 108a with respect to the remainder of cap body 102. Therefore, it is only upon application of a predetermined pressure (that is the result of a predetermined force) to moveable surface portion 108a of the target site contact surface 108 that movement from the first position to the second position is achieved. The predetermined force can be, for example, in the range of 2 Newtons to 20 Newtons.

Prior to use of cap 100, moveable surface portion 108a and moveable cap body portion 102a protrude beyond the remainder of target site contact surface 108 (see FIG. 2 in particular). Therefore, in use, moveable cap body portion 102a will make initial contact with the target site when cap 100 urged toward the target site. Such initial contact is depicted in FIG. 3A, wherein the target site is the end of a user's finger F.

During use of cap 100, target site contact surface 108, including moveable surface portion 108a, is urged against a target site (e.g., a dermal tissue target site of a user's finger) such that cap body 102 engages (i.e., contacts) the dermal tissue target site and a target-site bulge TB is created within opening 110 (see FIG. 3C). Such a target site bulge is expected to result in improved bodily fluid expression, as has been described in International Application PCT/US2003/036513 (published as WO 2004/045375 A2 on Jun. 3, 2004), which is hereby incorporated in full by reference.

When such urging is done with sufficient force, spring 120 is compressed. The spring constant of spring 120, thus, determines the force required to move moveable cap body portion 102a from the first position to the second position and the force constant can be predetermined such that adequate pressure is applied to the target site to engender a successful expression of bodily fluid sample upon lancing of the target site.

When moveable cap body portion 102a is in the second position (see FIG. 3C), electrical contact pads 112 and 114 are touching, thus completing an electrical circuit (not shown in the FIGs.) and, thereby, sending an electrical signal via electrical signal wire 116 to the lancing device. In this regard, electrical contact pads 112 and 114 and electrical signal wire 116 serve as a sensor that detects movement of moveable cap body portion 102a (and moveable surface portion 108a) into the second position and communicates such detection to the lancing device.

The lancing device can, for example, employ the electrical signal to immediately initiate lancing of a target site or to trigger a timer within the lancing device that serves to delay initiation of lancing. Such a delayed initiation can occur after a time interval (i.e., a delay) in the range of, for example, 0.5 seconds to 5.0 seconds. If desired, the time interval can be varied from use-to-use such that a user does not become accustomed to the time interval and prematurely withdraw the target site from the cap prior to lancing. Moreover, if cap 100 is withdrawn from the target site such that electrical contacts pads 112 and 114 no longer touch, then the timer can be reset and lancing delayed until, and if, electrical contact pads 112 and 114 again touch and the timer again triggered.

Further characteristics of caps according to embodiments of the present invention are evident from the sequence of FIGS. 3A, 3B and 3C, wherein cap 100 is depicted being urged against a target site (namely a dermal tissue target site TS on the distal end of a user's finger F) under a predetermined pressure. During use of cap 100, moveable surface portion 108a of moveable cap body portion 102a makes initial contact with dermal tissue target site TS (see FIG. 3A). Moreover, in the embodiment of FIG. 3A, moveable cap body portion 102a is depicted as making initial contact below the most distal knuckle (not shown) of the user's finger F.

As dermal tissue target site TS is further urged against cap 100, spring 120 becomes partially compressed (see FIG. 3B) and moveable cap body portion 102a applies a counterforce (i.e., a counter-pressure) against dermal tissue target site TS. This counterforce results in blood being (i) forced toward the user's fingertip and (ii) pressurized within the dermal tissue target site. In addition, compression of spring 120 results in electrical contact pad 112 moving closer to electrical contact pad 114 (as is evident from a comparison of FIGS. 3A and 3B).

Referring now to FIG. 3C, upon the application of a predetermined force to moveable surface portion 108a (for example, a force in the range of 15 Newton to 18 Newton), moveable surface portion 108a is placed into the second position depicted in FIG. 3C. In this second position, electrical contact pad 112 is in electrical contact with electrical contact pad 114 and moveable surface portion 108a is substantially aligned with the remainder of target site contact surface 108. In addition, the application of the predetermined force has created a target site bulge TB within aperture 110 of cap 100 (see FIG. 3C).

Cap 100 has several beneficial characteristics. For example, a user of a lancing device that incorporates cap 100 is not required to press a button or a switch to initiate lancing. Also, lancing is only initiated when a predetermined pressure has been applied to target site contact surface 108 (including moveable surface portion 108a) such that moveable cap body portion 102a has moved from the first position to the second position. The predetermined pressure can be predetermined such that it serves to express an adequate bodily fluid sample (for example, by the creation of a target site bulge with an opening of the cap body). In addition, if an optional timer is employed in the lancing device, lancing can be delayed as needed to optimize bodily fluid expression. Furthermore, caps according embodiments of the present invention include a sensor that is responsive to force (pressure) applied directly to a contact surface of the cap and, therefore, there is a minimal risk of erroneously sensing forces applied to components of the lancing device or to other surfaces of the cap.

A further benefit of caps according to embodiments of the present invention is a reduction in apparent pain associated with lancing. Initiating lancing upon sensing of adequate applied pressure is expected to increase the likelihood of lancet penetration to a proper penetration depth. A user is therefore less likely to have to re-lance due to improper penetration depth.

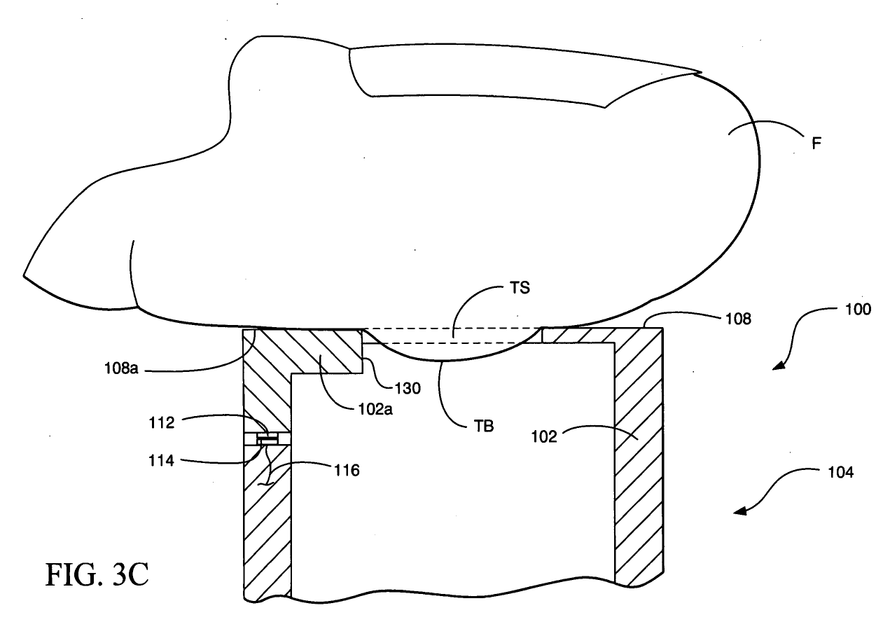

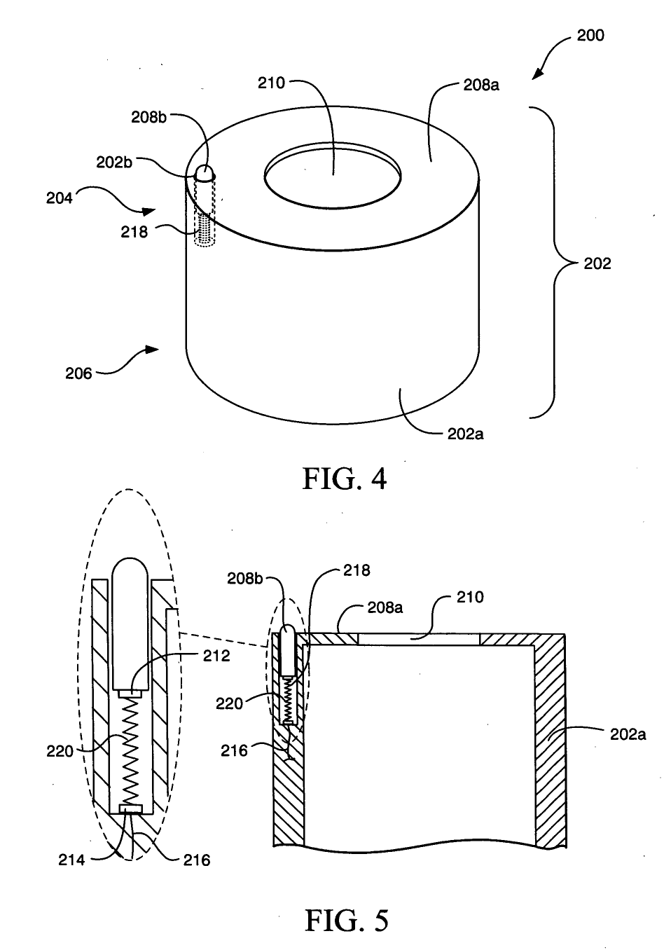

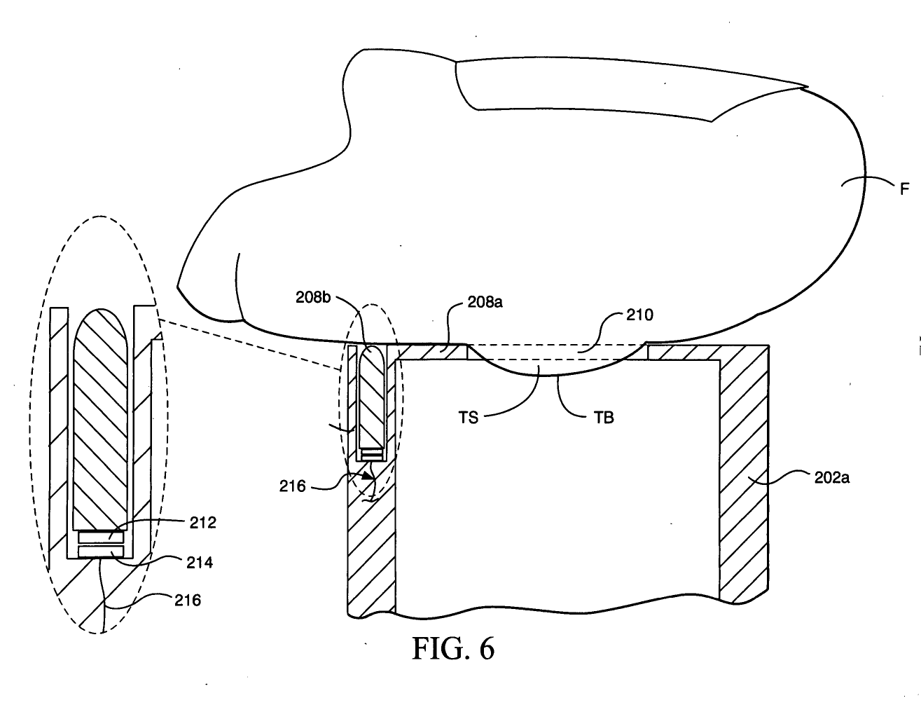

FIG. 4 is a simplified perspective view of a cap 200 for a lancing device (not shown in FIG. 4) according to another exemplary embodiment of the present invention. FIG. 5 is a combined simplified cross-sectional and partial enlarged view of the distal end of cap 200 and FIG. 6 is a simplified cross-sectional view of cap 200 urged against a target site with a predetermined pressure.

Referring to FIGS. 4, 5 and 6, cap 200 includes a cap body 202 (including cap body portion 202a and moveable cap body portion 202b described further below) with a distal end 204 and a proximal end 206. One skilled in the art will recognize that moveable cap body portion 202b is essentially a “nib” operatively engaged with cap body portion 202a.

Distal end 204 includes a target site contact surface 208a on cap body portion 202a and moveable contact surface 208b on moveable cap body portion 202b. Proximal end 206 can be configured for attachment to a lancing device (for example, to a housing of a lancing device) by a snap fit, frictional fit or other suitable attachment technique.

Cap body 202 has an opening (i.e., aperture) 210 therethrough that extends from proximal end 206 to distal end 204. Cap 200 further includes a sensor consisting of electrical contact pads 212 and 214 and electrical signal wire 216. One skilled in the art will recognize that the entire length of electrical signal wire 216 is not depicted in FIG. 5.

Moveable contact surface 208b is moveable between a first position (depicted in FIGS. 4 and 5) and a second position (depicted in FIG. 6) upon application of a predetermined force (pressure) to the moveable contact surface 208b. Referring to FIGS. 4 and 5, it should be noted that moveable contact surface 208b is a surface of moveable cap body portion 202b of cap body 202 and that moveable cap body portion 202b travels within a guide recess 218 of cap body 202.

Cap 200 further includes a spring 220 with a predetermined spring constant. Spring 220 is disposed between moveable cap body portion 202b and the remainder of cap body 202 within guide recess 218. Spring 220 is configured and adapted such that the force of spring 220 must be overcome to move moveable cap body portion 202b (and moveable contact surface 208b) from the first position of FIGS. 4 and 5 to the second position of FIG. 6. In other words, spring 220 serves to resiliently bias moveable cap body portion 202b with respect to the remainder of cap body 202. Therefore, it is only upon application of a predetermined pressure to moveable contact surface 208b that movement from the first position to the second position is achieved.

During use of cap 200, target site contact surface 208a and moveable contact surface 208b are urged against a target site (e.g., a dermal tissue target site TS of a user's finger F) such that cap body 202 engages (i.e., contacts) the dermal tissue target site and a target site bulge TB is created within opening 210 (see FIG. 6). Such a target site bulge is expected to result in improved bodily fluid expression.

When such urging is done with sufficient force, spring 220 is compressed. The spring constant of spring 220, thus, determines the force required for movement to occur between the first position and the second position. Therefore, the spring constant can be predetermined such that adequate pressure is applied to the target site to result in a successful expression of bodily fluid sample upon lancing of the target site.

When moveable cap body portion 202b is in the second position (see FIG. 6), electrical contact pads 212 and 214 are touching, thus completing an electrical circuit (not shown in the FIGs.) and, thereby, sending an electrical signal via electrical signal wire 216 to the lancing device. In this regard, electrical contact pads 212 and 214 and electrical signal wire 216 serve as a sensor that detects movement of moveable cap body portion 202b (and moveable contact surface 208b) into the second position and communicates such detection to the lancing device.

Cap body 202 is configured, therefore, to sense when a predetermined pressure is being applied to a target site (i.e., a predetermined applied pressure) and signal a lancing device accordingly. The lancing device can, for example, employ the electrical signal to immediately initiate lancing of a target site or to trigger a timer within the lancing device that serves to delay initiation of lancing.

Moveable cap body portion 202b of cap body 202 has a beneficially low risk of being accidentally depressed due to the relatively small size of moveable cap body portion 202b and its disposition on the distal end 204 of cap body 202. This reduces a likelihood of launching a lancet within the lancing device by accidental depression of moveable cap body portion 202b.

It should be noted that caps according to the present invention can employ multiple sensors and multiple moving cap body portions in order to determine whether or not a predetermined applied pressure is being applied at multiple locations on a distal end contact surface. In other words, the sensors can be sensors uniformly distributed about the cap body. For example, the embodiment of FIGS. 4, 5 and 6 can be modified to include multiple “nibs,” each in a guided recess, and associated electrical contact pads and electrical signal wires disposed symmetrically about the circumference of cap body 202.

Once apprised of the present disclosure and the embodiments of FIGS. 1-6, one skilled in the art will readily recognize that caps according to various embodiments of the present invention generally include a cap body and at least one sensor. In addition, the cap body includes a distal end with a target site contact surface, a proximal end for attachment to a lancing device and an opening through the cap body from the distal end to the proximal end. Furthermore, at least a portion of the target site contact surface is moveable between a first position and a second position upon application of a predetermined pressure to that portion of the distal end contact surface, and the sensor is configured to detect movement of the portion of the distal end contact surface into the second position and communicate such detection to the lancing device.

A kit according to embodiments of the present invention includes a lancing device (as described herein) and a cap for the lancing device. Moreover, such a cap includes a cap body and at least one sensor. In addition, the cap body includes a distal end with a target site contact surface, a proximal end for attachment to a lancing device and a opening through the cap body from the distal end to the proximal end. Furthermore, at least a portion of the target site contact surface is moveable between a first position and a second position upon application of a predetermined pressure to the portion of the distal end contact surface, and the sensor is configured to detect movement of the portion of the distal end contact surface into the second position and communicate such detection to the lancing device.

If desired, the lancing device can include a timer and the sensor can communicate such detection to the timer. The cap can be any cap as described herein. Moreover, although particular sensor embodiments have been described, the sensor of caps and kits according to embodiments of the present invention can be any suitable sensor, including mechanical sensors, electrical sensors, optical sensors and combinations thereof. In addition, the lancing device can be adapted to employ artificial learning to determine and utilize an optimal time interval between receiving a communication from the sensor and initiating lancing of the target site. Such adaptation can be accomplished, for example, by incorporating a suitably programmed microprocessor into the lancing device.

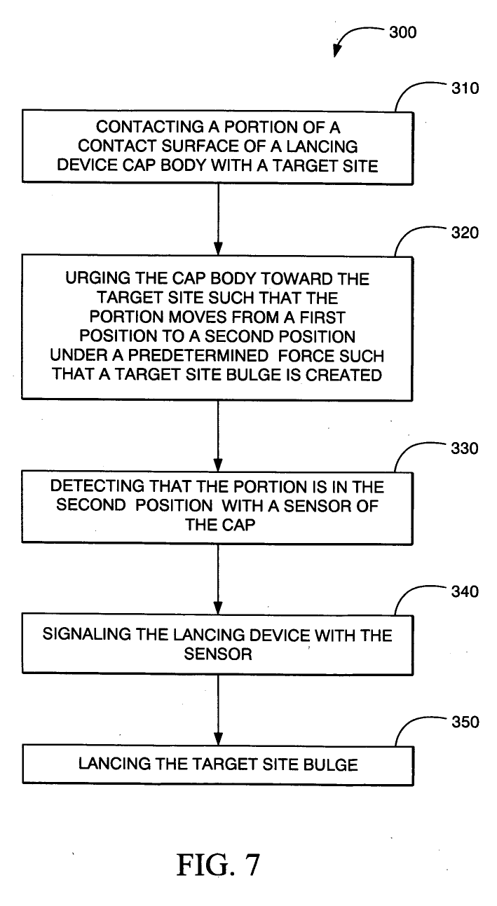

FIG. 7 is a flow diagram depicting stages in a method 300 for lancing a target site (e.g., a dermal tissue target site on a user's finger) according to an exemplary embodiment of the present invention. Once apprised of the present disclosure, one skilled in the art will recognize that method 300 can be, for example, accomplished and using caps and kits according to various embodiments of the present invention and can include techniques associated with such caps and kits as described herein.

Method 300 includes contacting at least a portion of a contact surface of a distal end of a cap body of a cap for a lancing device with a target site, as set forth in step 310. In step 310, the cap body has a distal end with a target site contact surface, a proximal end for attachment to a lancing device and an opening through the cap body from the distal end to the proximal end of thereof. In addition, the cap has at least one sensor.

Moreover, in method 300 at least a portion of the target site contact surface is moveable between a first position and a second position upon application of a predetermined pressure (force) to the portion of the distal end contact surface, and the sensor is configured to detect movement of the portion of the distal end contact surface into the second position and communicate such detection to the lancing device.

Subsequently, at step 320, the cap body is urged towards the target site such that the portion of the contact surface moves from a first position to a second position under the predetermined force and a target site bulge is created within the opening of the cap body. The sensor is then employed to detect that the portion of the contact surface is in the second position (see step 330) and signals the lancing device upon such detection (see step 340). The target site bulge is thereafter lanced with the lancing device.

It should be understood that various alternatives to the embodiments of the invention described herein may be employed in practicing the invention. It is intended that the following claims define the scope of the invention and that methods and structures within the scope of these claims and their equivalents be covered thereby.

That which is claimed is:

1. A method for lancing a target site, the method comprising:
contacting at least a portion of a contact surface of a distal end of a cap body of a cap for a lancing device with a target site, wherein the cap for a lancing device includes:
a cap body with
a distal end with a target site contact surface;
a proximal end for attachment to a lancing device;
a opening through the cap body from the distal end to the proximal end of thereof and
at least one sensor,
wherein, at least a portion of the target site contact surface is moveable between a first position and a second position upon application of a predetermined pressure to the portion of the distal end contact surface, and
wherein the sensor is configured to detect movement of the portion of the distal end contact surface into the second position and communicate such detection to the lancing device;
urging the cap body towards the target site such that the portion of the contact surface moves from a first position to a second position under a predetermined applied pressure and a target site bulge is created within the cap body;
detecting, with the sensor, that the portion of the contact surface is in the second position;
signalling the lancing device that the portion of the contact surface is in the second position; and
lancing the target site bulge with the lancing device.

2. The method of claim 1, wherein the target site is a dermal tissue target site.

3. The method of claim 1, wherein the predetermined applied pressure is the result of applying a force in the range of from about 2 Newton to about 20 Newton.

4. The method of claim 1, wherein the lancing step is initiated following a delay after the signalling step.

5. The method of claim 4, wherein the delay is in the range of from about 0.5 seconds to 5 seconds.

6. The method of claim 4, wherein the delay is ascertained by a timer of the lancing device.

7. The method of claim 4, wherein the delay is varied from use-to-use of the lancing device.

8. The method of claim 4, wherein the delay has been optimized by artificial learning.

9. The method of claim 1, wherein the target site is a dermal tissue target site of a user's finger.

10. The method of claim 9, wherein the predetermined applied pressure is the result of applying a force in the range of from about 15 Newton to about 18 Newton.
